(19) United States
(12) Patent Application Publication
(10) Pub. No.: US 2007/0038309 A1
(43) Pub. Date: Feb. 15, 2007

(54) METHODS AND DEVICES FOR SUPRAGLOTTIC SECRETION DIVERSION

(75) Inventor: AMIR ABOLFATHI, Woodside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: APMed Solutions, Inc., Portola Valley, CA (US)

(21) Appl. No.: 11/428,640

(22) Filed: Jul. 5, 2006

Related U.S. Application Data
(60) Provisional application No. 60/707,416, filed on Aug. 10, 2005.

(51) Int. Cl. A61F 2/04

(52) U.S. Cl. 623/23.68

(57) ABSTRACT

A supraglottic diversioned device comprises a body and structure for diverting secretions, food, liquids, and other non-gaseous materials away from the trachea into the patient's esophagus. The body is adapted for implantation above the larynx at the bifurcation between the trachea and the esophagus. Optionally, valve structure can be provided, particularly to open upon exhalation. In all cases, the device will have air flow paths for both inhalation and exhalation.

### CROSS-REFERENCES TO RELATED APPLICATIONS

The present application claims the benefit of provisional U.S. Application No. 60/707,416 (Attorney Docket No. 025808-000300US), filed Aug. 10, 2005, the full disclosure of which is incorporated herein by reference.

### BACKGROUND OF THE INVENTION

1. Field of the Invention

The present invention relates generally to medical methods and apparatus. More particularly, the present invention relates to devices and methods for diverting foods, liquids, and secretions from the trachea to the esophagus in a breathing patient to limit or prevent passage of the materials into the patient's lungs.

Aspiration pneumonia is a common but serious condition which can arise from a number of causative factors. Of particular interest to the present invention, aspiration pneumonia can result from obstruction of the lower airways caused by aspiration of fluids, solids, secretions, and other particulate matter in unconscious or semi-conscious patients. While acute obstructions can be treated by tracheal suction, long-term treatment and prevention is more difficult.

For these reasons, it would be desirable to provide methods and apparatus for inhibiting the passage of fluids and particulates which would otherwise enter a patient's trachea and pass into the lower airways. It would be particularly desirable if such methods and apparatus allowed for substantially unimpaired breathing while permitting substantially normal speech. At least some of these objectives will be met by the inventions described hereinafter.

2. Description of the Background Art

U.S. Pat. No. 6,840,242 describes a tracheostomy aspiration suction tube for use with or without a tracheostomy cuff. Other tracheostomy tubes are described in U.S. Pat. Nos. 6,612,305; 6,575,944; 6,460,540; 5,957,978; 5,653,231; 5,392,775; 5,107,828; 5,056,515; 5,054,484; 4,979,505; 4,280,492; 4,278,081; and published U.S. application 2003/0037789. Certain endotracheal tubes are described in U.S. Pat. Nos. 6,843,250; 5,501,215; 5,311,864; 5,143,062; 5,067,497; 4,840,173; and 4,305,392.

### BRIEF SUMMARY OF THE INVENTION

The present invention provides devices, methods, and systems for inhibiting the entrance of foods, liquids, secretions, particulates, and other materials into a patient's trachea. In particular, the methods and devices provide a diversion structure which allows air flow therethrough so the patient can inhale, exhale, and speak while preferentially deflecting liquid and solid materials away from the trachea and into the patient's esophagus.

In the first specific embodiment, a supraglottic secretion diversion device comprises a body and means on the body for diverting liquid and solid materials away from the patient's trachea. The body is implantable above the larynx at the bifurcation between the trachea and the esophagus. The body is adapted to remain implanted at this location, often being adapted to be sutured in place. Alternatively, the body can be constructed to be self-expanding so that it may be placed at the bifurcation in a radially constrained configuration and allowed to expand in situ to anchor in place.

The secretion diversion means will typically comprise a non-linear flow path within the body which permits air flow in both directions but which diverts at least most non-gaseous materials into the esophagus. Optionally, the device may comprise a one-way or two-way valve structure which at least partially closes when the patient is not inhaling or exhaling. In particular embodiments, such valve structures may comprise a dome structure including a plurality of interleaved leaves and which allow inhaling through interstices between adjacent leaves. In a second specific embodiment of the valve structure, the body has a hinged flap which opens in response to exhaling and a slotted bypass which allows inhaling even when the hinged flap is closed.

In another aspect of the present invention, methods for diverting liquids and solids, and other non-gaseous materials in a breathing patient comprise implanting a secretion diversion device above the larynx at the bifurcation between the trachea and the esophagus. The device is adapted to divert secretions away from the trachea and into the esophagus while allowing relatively unimpeded inhalation and exhalation by the patient. In specific examples of the method, the various examples of the diversion device described above may be implanted into the patient.

### BRIEF DESCRIPTION OF THE DRAWINGS

FIGS. 1A and 1B illustrate a first embodiment of the flow diverting device of the present invention comprising a plurality of interleaved leave elements.

FIGS. 2 and 3 illustrate implantation of the device of FIGS. 1A and 1B at the bifurcation between the trachea and esophagus of a patient.

FIGS. 4A and 4B illustrate a second embodiment of the flow diverting device of the present invention comprising a hinged flap on a slotted cylindrical body.

FIGS. 5 and 6 illustrate implantation of the device of FIGS. 4A and 4B at the bifurcation between the esophagus and the trachea of a patient.

### DETAILED DESCRIPTION OF THE INVENTION

The present invention is intended primarily to provide for a long term implantable device which diverts foods, liquids, secretions and other non-gaseous materials which might otherwise enter a patient's trachea and potentially cause aspiration pneumonia or other difficulties into the patient's esophagus. The structures are adapted to be implanted in the supraglottic region immediately above the larynx and at the bifurcation between the esophagus and the trachea. The devices may comprise passive diversion devices, e.g. including a labyrinth which allows the passage of air while inhibiting the passage of non-gaseous medium. Alternatively, the devices may comprises active valve structures which open and close in response to inhalation and/or exhalation of the patient.

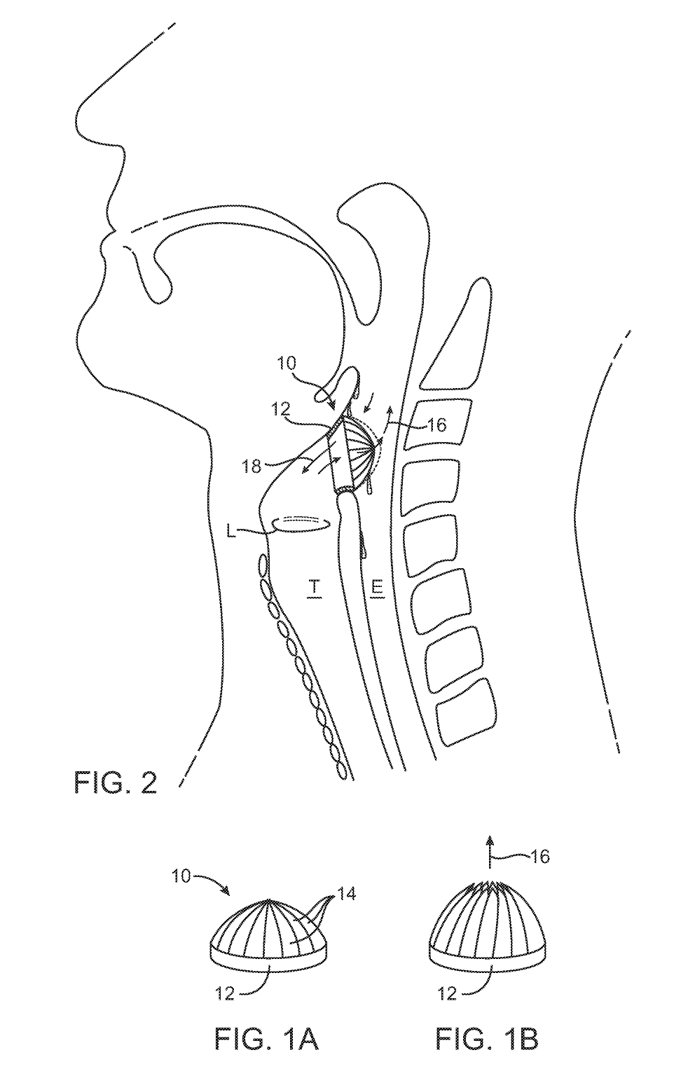

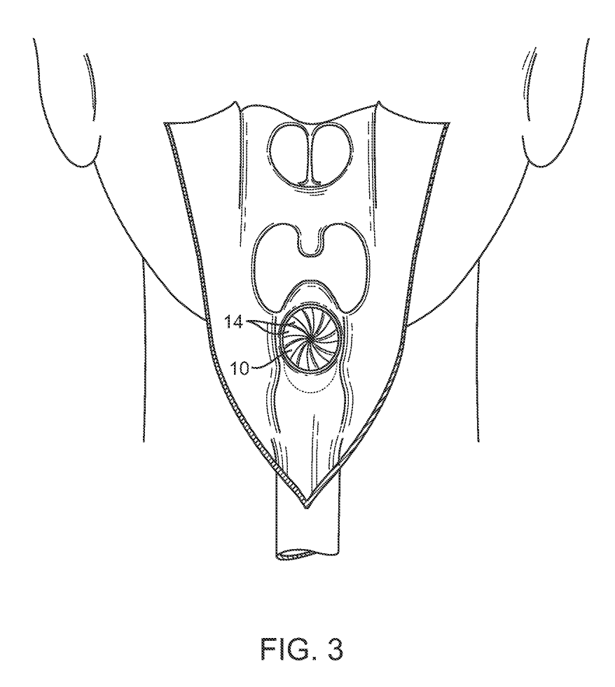

Referring now to FIGS. 1A and 1B, a first diversion structure 10 comprises a base 12 and a plurality of interleaved leave elements 14. The leaf elements are formed generally as a dome, and the individual leaves will open, as shown in FIG. 1B, in response to pressure causing air flow through the structure in the direction of arrow 16. Base 12 may be adapted for suturing in place or may be resilient so that it may be collapsed for introduction and allowed to self-expand an anchor in place in the patient, generally shown in FIG. 2.

As shown in FIG. 2, exhalation air flows in the direction of arrows 16 causes the leaves 14 of diversion structure 10 to open, as shown in broken line. In contrast, inhalation in the direction of arrows 18 does not cause the leaves to open, but rather will flow through naturally interstices between the leaves when in the closed configuration of FIG. 1A. In this way, the patient can continue to breath while the structure generally remains closed to inhibit the flow of secretions, solid materials, food, drink, and the like, from entering the trachea T while being diverted to the esophagus E. As the device is above the patient's larynx L, the patient will continue to be able to speak, aided by the opening of the leaves as the patient exhales and speaks.

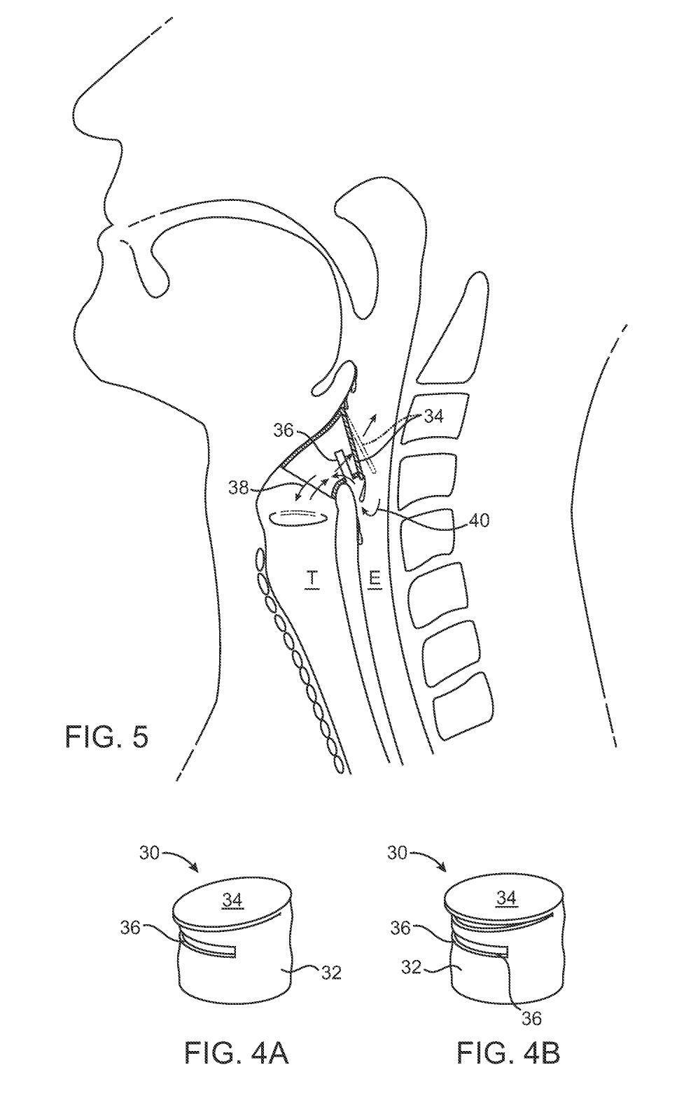

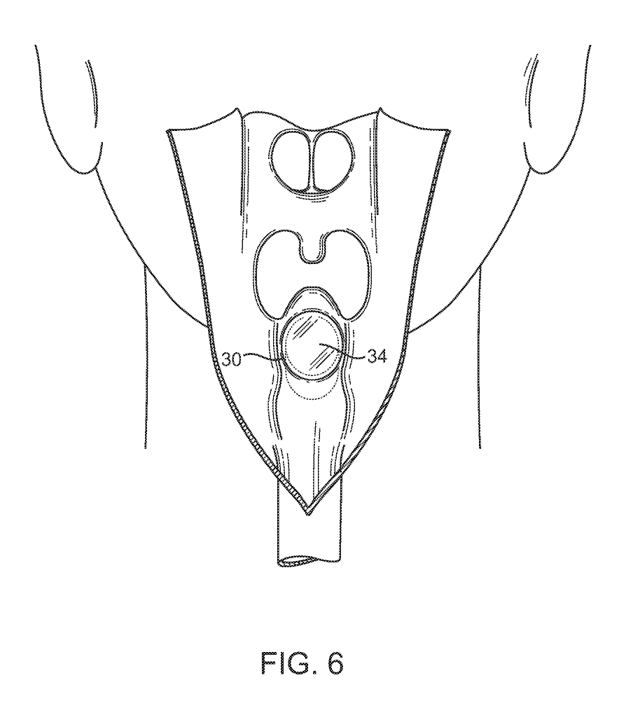

An alternative flow diversion device 30 is shown in FIGS. 4A and 4B and comprises that cylindrical body 32 having a hinged flap 34 at one end thereof. A slot 36 is formed as a bypass in the body 32. Body 32 may be adapted for suturing in place at the bifurcation between the trachea T and esophagus E, generally as shown in FIGS. 5 and 6. Alternatively, the body may be resilient and self-expanding, as generally described above.

When in place, the flap valve 34 will generally open when the patient is exhaling, as shown by arrows 38. In contrast, inhalation can take place through slot 36 in the direction shown by arrows 40.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A supraglottic secretion diversion device comprising;
a body implantable above the larynx at the bifurcation between the trachea and the esophagus;
means on the body for diverting secretions away from the trachea into the esophagus while allowing relatively unimpeded inhalation and exhalation through the body.

2. A device as in claim 1, wherein the body is adapted to be sutured in place.

3. A device as in claim 1, wherein the body is self-expanding so that it may be placed in a radially constrained configuration and expand in situ to anchor in place.

4. A device as in claim 1, wherein the means comprises a non-linear flow path that permits air flow in both directions but diverts at least most non-gaseous materials into the esophagus.

5. A device as in claim 1, wherein the device comprises a one-way or two-way valve structure which at least partially closes when the patient is not inhaling or exhaling.

6. A device as in claim 5, wherein the valve structure comprises a dome structure including a plurality of leaves which open in response to exhaling and which allow inhaling through interstices between adjacent leaves.

7. A device as in claim 5, wherein the valve structure comprises a hinged flap which opens in response to exhaling and a slotted bypass that allows inhaling.

8. A method diverting secretions in a breathing patient, said method comprising:
implanting a secretion diversion device above the larynx at the bifurcation between the trachea and the esophagus, said device diverting secretions away from the trachea into the esophagus while allowing relatively unimpeded inhalation and exhalation by the patient.

9. A method as in claim 8, wherein implanting the devices comprises suturing the device in place.

10. A method as in claim 8, wherein implanting the device comprises expanding the device in situ to anchor in the trachea above the larynx.
